(19) United States
(12) Patent Application Publication
(10) Pub. No.: US 2006/0286130 A1
(43) Pub. Date: Dec. 21, 2006

(54) Sterile gelling agents

(76) Inventors: Prabhakar Harshal Bhagwatwar, Pune (IN); Balkrishna Mahesh Paithankar, Kopergaon (IN); Pradeep Sharma, Mumbai (IN); Varada Ramesh Bapat, Aurangabad (IN); Alibhai Ismail Mathakiya, Garida (IN); Shahajirao Chandrashekhar Kadam, Aurangabad (IN); Subhash Bhushan Yeola, Aurangabad (IN)

Correspondence Address: BIO INTELLECTUAL PROPERTY SERVICES (BIO IPS) LLC, 8509 KERNON CT., LORTON, VA, 22079, US

(21) Appl. No.: 11/155,112

(22) Filed: Jun. 17, 2005

(51) Int. Cl. A61K 47/00; A61K 9/00

(52) U.S. Cl. 424/400; 514/785

(57) ABSTRACT

This invention is directed to sterile gelling agents, which retain their physico-chemical properties so that they can be used in drug delivery systems. Also described are the processes for obtaining a sterile gelling agent(s).

### FIELD OF THE INVENTION

This invention is directed towards a sterile gelling agent and a process for the manufacture of the gelling agent. The sterile, gelling agent may further be incorporated into delivery systems preferably drug delivery systems.

### BACKGROUND OF THE INVENTION

Conventional liquid formulations have been used in the past for the parenteral administration of bioactive agents for the treatment of a variety of disease conditions in human beings and animals. Such formulations include simple aqueous or non-aqueous solutions or suspensions, lyophilized powders for reconstitution for administration via routes such as intravenous, intraarterial, subcutaneous, intramuscular and the like; solid implants for subdermal administration; microencapsulated products for intramuscular administration and the like. More recently, research has focused on the development of controlled release compositions which form the delivery systems inside the body after administration. Such compositions include the ATRIGEL system described by Dunn et al. (4,938,763), the ReGel system developed by Rathi et al. (PCT Application WO 00/18821) and others. Even more recently, delivery compositions which form microcarriers inside the body have been developed to overcome the problems associated with the delivery systems described above. Such problems include prolonged processing times, use of costly equipments, use of toxic and often carcinogenic organic solvents and subsequent problems associated with their removal from the composition and the like. These and other problems are overcome by in-situ microcarrier forming delivery compositions as described by Bhagwatwar et al. (U.S. 20030049320 A1, AU 0222505 A5 and WO 02/49573 A3).

All of these compositions are required to be sterile, endotoxin and foreign particulate free to reduce the probability of infections to the animal because of contamination by maicroorganisms. The term “Foreign particulate free” is meant to indicate the absence of any particulate matter, which is not supposed to be there in the composition and excludes drug particles, controlled release microparticulates and the like. Conventional methods for the manufacture of sterile compositions include sterilization by moist heat (autoclaving), sterilization by dry heat, ethylene oxide sterilization (gaseous sterilization), exposure to ultraviolet rays or to gamma irradiation or sterilization by aseptic processing. These and other methods of sterilization are described in detail in Pharmaceutical Dosage Forms: Parenteral Medications (Eds. Avis, Lachman and Lieberman, Volumes 1-3).

Most of the conventional formulations for parenteral administration described above can be readily processed by one of the manufacturing processes mentioned above and the choice of an appropriate method for their sterilization is within the scope of understanding of a person of ordinary skill in the art of manufacture of parenteral dosage forms. Thus, solution formulations of bioactive agents which are stable to temperature can be readily autoclaved post-processing of the formulation and filling into the final container. This process known as “terminal sterilization” is generally used for ensuring sterility of large volume parenterals such as normal saline, dextrose saline and the like. For temperature labile bioactive agents the formulations have to necessarily be processed aseptically that is through filtration through sterilizing grade filters which have a nominal pore size of 0.22 μm. Similarly, such agents which are sensitive to temperature and also to water can also be filled into vials, ampoules or syringes and then lyophilized. Lyophilized products which are free from moisture are then reconstituted before administration providing a prolonged shelf-life. Other compositions can be sterilized by ethylene oxide or by irradiation. Each of these methods suffers from disadvantages such as residual ethylene oxide, degradation due to heat or irradiation and others.

It is especially difficult to manufacture sterile controlled release products for parenteral administration such as the microencapsulated products, in-situ forming implants and the in-situ microcarrier forming gelled polymeric dispersions. Of all of the controlled release products mentioned above, sterile processing of the in-situ microcarrier forming gelled polymeric dispersions poses the greatest challenge to the formulation scientist because of the complex nature of the delivery composition. These gelled polymeric dispersions are comprised of dispersions of organic solvent solvent of biocompatible polymers in a continuous oleaginous phase gelled and stabilized by emulsifiers chosen from sorbitan monostearate or monopalmitate. The organic solvents are preferably water-soluble though water-immiscible solvents can also be used. Upon coming in contact with an aqueous medium, the oily continuous phase gets emulsified and the polymers from the droplets precipitate through the mixing and extraction of water-soluble solvents from the droplets. The detailed composition and processes to make the compositions are described in U.S. 20030049320 A1, AU 0222505 A5 and WO 02/49573 A3 to Bhagwatwar et al. and are all incorporated herein by reference.

Though different methods to prepare sterile compositions are described in the literature, the literature is silent with respect to sterile in-situ microcarrier forming gelled polymeric dispersions and processes to manufacture such compositions. Problems associated with the sterile processing of the in-situ microcarrier forming gelled polymeric dispersion compositions include: instability of the polymer to heat, moisture and gamma irradiation, difficulty of aseptic processing of drug-free or drug-containing polymer solutions of high concentrations of greater than 40% w/w of polymer and upto 50% w/w of bioactive agent with respect to the polymer, and preparation of the sterile gelling agent bulk sorbitan monostearate or sorbitan monopalmitate.

The gelling agents of this invention as commercially available are not free from foreign particulate material and contain significant quantities of impurities which add color to the final product making it unacceptable for parenteral use. Further, the use of water for the processing of these gelling agents is not feasible because the gelling agents would degrade during autoclaving for example resulting in a loss of gelling capability. Also, presence of moisture in any of the materials, specially the gelling agent would result in the loss of physical stability of the gelled dispersions.

A process to manufacture sterile in-situ microcarrier forming gelled polymeric dispersion compositions would be a tremendous improvement in the current state-of-the-art in the development of commercial products using these compositions.

There is thus a need for preparing a sterile bulk sorbitan monostearate or sorbitan monopalmitate gelling agent substantially free from moisture and foreign particulate matter thereby improving its utility value as a pharmaceutical entity.

There is a further need to provide a process for the manufacture of sterile in-situ microcarrier forming gelled polymeric dispersion compositions containing bioactive agents for their immediate or controlled release.

There is also a need to provide such sterile in-situ microcarrier forming gelled polymeric dispersion compositions for use in the treatment of various disease conditions in human beings and animals.

### SUMMARY OF THE INVENTION

A novel method for the manufacture of sterile gelling agent for use in a suitable composition or delivery system for therapeutic administration is described. The method involves the use of aseptic processing alone or gamma-irradiation alone or a combination of aseptic processing and gamma irradiation to achieve a product of the desired attributes including sterility, freedom from foreign particulates, syringeability, particle formation upon coming in contact with aqueous media, potency of active, and physical stability.

The present inventors have surprisingly found that the manufacture of a sterile drug delivery system, with the desired characteristics is dependant on the successful preparation of a sterile gelling agent bulk which is free from foreign particulate matter and substantially free from moisture, while retaining its physicochemical characteristics like its capability to gel and physically stabilize the gelled polymeric dispersion composition if the sterile gelling agent be incorporated in a gelled polymeric drug delivery system.

The gelling agents of the invention include sorbitan monostearate or sorbitan monopalmitate which are known to be soluble in a variety of organic solvents (both volatile and non-volatile) at elevated temperatures followed by gelation of the solvents upon cooling. This principle has been used in the development of the in-situ microcarrier forming gelled polymeric dispersion compositions by Bhagwatwar et al. (U.S. 20030049320 A1, AU 0222505 A5 and WO 02/49573 A3). The present inventors further have also astonishingly observed that certain volatile organic solvents such as ethanol, methanol and the like, not only act as good solvents for the gelling agents at elevated temperature but are readily removable through simple vacuum drying, with or without application of heat, without any change in the product characteristics. Further such solvents also ensure the sterility of the finished bulk. In addition, a majority of the solvent can be removed through the addition of a non-solvent such as for example water for injection. One other property of the use of such solvents includes that when excess solvent is added to the gelled bulk upon cooling, the sterile bulk gelling material precipitates out. Also, the residual solvent, if any remains after this rigorous processing, is non-toxic, as the solvents such as ethanol are acceptable for parenteral administration.

In another embodiment of the invention, a sterile gelling agent is prepared by subjecting a foreign particulate free gelling agent as processed above to gamma irradiation.

In another aspect of the invention a sterile polymer-drug solution is prepared by gamma irradiation of such a solution.

In yet another aspect of the invention, a in-situ microcarrier forming gelled polymeric dispersion composition prepared using the gelling agents treated as above are subjected to gamma irradiation to prepare a sterile product useful for human administration without any significant loss in potency or behavior of the gelled dispersion composition in physical stability, syringeability or particle formation upon coming in contact with aqueous media.

Another embodiment of the invention describes the administration of this sterile comrposition to an animal species.

These and other embodiments of the invention are described in greater detail in the further sections of this application.

### DETAILED DESCRIPTION OF THE INVENTION

Preparation of a Sterile Gelling Agent

The gelling agents of the invention include sorbitan monostearate and/or sorbitan monopalmitate. Both of these materials are available from a number of suppliers commercially for topical or oral use. Sterile material free from foreign particulates is not available and is difficult to produce. It is now possible to produce such sterile, foreign particulate free material through the use of aseptic precipitation and drying alone or in conjunction with gamma irradiation. It is essential that the process used for the preparation of a sterile bulk does not introduce any change in physicochemical properties of the gelling agent which will in turn cause a change in the gelling potential of the gelling agent.

The process to provide a foreign particulate free, sterile bulk gelling agent comprises the steps of:
1. Dissolution of the gelling agent(s) in volatile organic solvents, if required at an elevated temperature, followed by filtration of the solutions also at the elevated temperature and subsequent evaporation to obtain a dry product OR
2. Dissolution of the gelling agent(s) in water-miscible organic solvents at an elevated temperature followed by filtration of the solutions also at the elevated temperature, precipitation of the gelling agents from solution either through addition of a non-solvent or cooling the solution to a lower temperature to cause phase-separation, filtration to recover the wet bulk and subsequent evaporation to obtain a dry product AND
3. Subjecting the foreign particulate-free gelling agent obtained from steps 1 and 2 to sterilization by gamma irradiation

The gelling agents of the invention viz. sorbitan monostearate or sorbitan monopalmitate are soluble in a variety of organic solvents including volatile organic solvents such as methylene chloride, ethyl acetate, benzene, petroleum ether, carbon tetrachloride, methanol, acetonitrile, acetone, ethanol, tetrahydrofuran and other volatile solvents, at elevated temperatures. Non-volatile organic solvents in which the gelling agents are soluble include NN′-dimethylacetamide (DMA), dimethylsulfoxide (DMSO), N-methyl-2-pyrrolidone MP), triacetin, triethyl citrate, benzyl alcohol, propylene carbonate, decylmethylsulfoxide, dimethylformamide (DMF), glycofural, benzoyl benzoate, alkyl esters of aromatic acids, polyethylene glycols (PEG), propylene glycol and the like, also at elevated temperatures.

The solvents for the invention should be biocompatible and not pose toxicity issues. Such solvents may be completely water-miscible, partially water-miscible or completely water-immiscible. Water-immiscible organic solvents such as dichloromethane, chloroform, ether, benzene, hexane and the like though otherwise used in the preparation of pharmaceutical compositions are generally toxic and require exotic methods such as lyophilization and the like for removal. Also, there are very strict guidelines for the levels of such residual solvents allowable in pharmaceutical compositions, especially so for parenteral administration. Water-miscible organic solvents are preferred. More specifically, such water-miscible organic solvents are chosen from DMA, DMSO, NMP, triacetin, triethyl citrate, benzyl alcohol, propylene carbonate, decylmethylsulfoxide, DMF, glycofural, benzoyl benzoate, alkcl esters of aromatic acids, PEG, propylene glycol, isopropanol, methanol, acetonitrile, acetone, ethanol, tetrahydrofuiran and the like. A specially preferred volatile water-miscible solvent is ethanol because of its proven antiseptic properties, its complete water-miscibility, its volatile nature, its biocompatibility and low toxicity potential, its solvating capability for the gelling agents of the invention at elevated temperatures and its known use in the pharmaceutical industry for parenteral administration of solution dosage forms. Preferred non-volatile water-miscible oraganic solvents include DMA, DMSO, DMF, NMP, PEG among others.

In one embodiment of the novel process of the invention, the sterile bulk gelling agent is prepared by dissolution in volatile water-miscible organic solvents specifically chosen from ethanol, methanol, acetonitrile, acetone, ethanol, tetrahydrofuran at a temperature above ambient, filtered through a sterilizing grade filter membrane at the elevated temperature and dried under vacuum at the elevated temperature.

In another aspect of this embodiment of the invention the solution of the gelling agent which has been filtered through a sterilizing grade filter membrane, is cooled to room temperature or lower to cause gelation of the solvent and the same solvent is added to the gel to cause precipitation of the gelling agent. This suspension is then filtered through a further sterilizing grade filter membrane and the wet mass which is substantially free from the organic solvent is then dried under vacuum at an elevated temperature. This procedure has the advantage of removal of a large percentage of the organic solvent from the gelled bulk which would otherwise be lost under vacuum. This solvent can then further be used in processing of a separate lot of the gelling agent. The term “substantially free” indicates that a large percentage of the organic solvent is removed when compared with the situation where all of the solvent is bound to the gelling agent in the form of a gel.

In a further embodiment of the invention, the solution of the gelling agent which has been filtered through sterilizing grade filter membranes at elevated temperatures is then added to a non-solvent to cause precipitation of the gelling agent. The non-solvent can be an organic solvent or can be water. The precipitated gelling agent is then treated further as in the earlier embodiment described above with the same advantages.

In a further aspect of this embodiment, the water-miscible organic solvent is non-volatile chosen from DMA, DMSO, NMP, triacetin, triethyl citrate, benzyl alcohol, decylmethylsulfoxide, DMF, glycofural, PEG, propylene glycol and the like. Upon precipitation from a non-solvent such as water, the gelling agent is subjected to drying to remove water. The residual organic solvent should be chosen advantageously to be the same as the one to be used in the final gelled polymeric dispersion.

In an additional embodiment of the invention, the sterile dried bulk gelling agent is subjected to sterilization by gamma irradiation. Surprisingly, the gelling agents are not degraded by this procedure. Other materials in the gelled polymeric dispersion can be processed by other techniques as described above and generally known in the art of preparation of sterile dosage forms.

The concentration of the gelling agent in the water-miscible solvent can be from 5% w/w to 80% w/w, preferably from about 10% w/w to 70% w/w and even more preferably from about 20% w/w to 60% w/w. The concentration of the gelling agent in the final solution will be dependent on the solvent chosen, the gelling agent solvent interactions, the processing temperature and of course the solubility of the gelling agent in the solvent. Of particular importance is the concentration of the gelling agent in the solvent, which allows filtration through a sterilizing grade membrane filter. The higher the concentration of the gelling agent in the solution the greater the yields and smaller the amounts of the expensive solvents that are used.

When a solvent/non-solvent type of precipitation is to be used, the ratio of the solvent to the non-solvent may be adjusted so that complete precipitation occurs to ensure maximum yields.

The temperature of preparation of the solution and also the processing will of course depend on the solvent to be used with a temperature below the boiling point of the solvent being preferred. Thus, for example, for a solvent such as ethanol a clear solution can be prepared at a temperature of 35-40° C. and higher at concentrations as high as 50% w/w, which can be filtered through a sterilizing grade 0.22 μn filter at the same temperature. But, for a solvent such as DMA, a solution with the same concentration needs to be processed at a higher temperature because of the viscosity imparted by the gelling agent.

The gelling agents of the invention are known to gel the solvents of the invention at high concentrations. Each gelling agent-solvent system will have a different temperature range at which the gelation occurs which will also -affect the processability of the solution.

The sterilizing grade filter can be any membrane, which has the capability to remove foreign particulates and also microorganisms to ensure sterility. Such filters usually have a pore size of 0.22 μm. Any membrane filter is acceptable for the practice of this invention as long as it can filter the solution and is compatible with the solvents of the invention. Such membranes include those made from nylon 66, cellulose acetate, cellulose nitrate, polytetrafluoroethylene (PTFE), silver membrane, gold membrane, polysulfone, polycarbonate and other known in the art and supplied by various manufacturers. The sterilizing grade filter could be preceded by a cleaning filter such as a 0.45 μm, 5 μm or 8 μm filter which can take up much of the burden from the sterilization filter. The choice of filters is within the scope of a person skilled in the art of development of pharmaceutical injectable dosage forms.

Any mode of filtration is acceptable as long as a sterile product is produced. Such methods include, vacuum filtration, filtration under positive pressure using compressed air or nitrogen and the like. Also, the use of cartridge filters or filter candles and the like are well within the scope of this description.

The drying of the sterile gelling agent bulk obtained after the above described processes can be conducted by any means known in the art, including for example tray drying in an oven with or without the application of vacuum and with or without heating, lyophilization, simple vacuum drying and other methods known to a person skilled in the art of processing pharmaceutical dosage forms. Where heating is required it is preferable to heat at a temperature above the boiling point of the solvent to be evaporated to ensure complete removal of the organic solvent.

Further, for the preparation of a sterile in-situ microcarrier forming gelled polymeric dispersion composition, the preparation of sterile solvents, polymers, oil and bioactive agents can be as per known procedures in the art such as aseptic filtration of the solvents and oil, gamma irradiation of the polymer or aseptic filtration of the polymer solution in a volatile organic solvent followed by evaporation of the solvent and the like and are well known to persons skilled in the art of manufacture of parenteral controlled release dosage forms.

The bioactive agents which can be incorporated into the sterile in-situ microcarrier forming gelled polymeric dispersion compositions can be chosen from peptide drugs, protein drugs, desensitizing agents, antigens, vaccines, anti-infectives, antibiotics, antimicrobials, antineoplastics, antitumor, antiallergenics, steroidal anti-inflammatory agents, analgesics, decongestants, miotics, anticholinergics, sympathomimetics, sedatives, hypnotics, antipsychotics,-psychic energizers, tranquilizers, androgenic steroids, estrogens, progestational agents, humoral agents, prostaglandins, analgesics, antispasmodics, antimalarials, antihistamines, cardioactive agents, non-steroidal anti-inflammatory agents, antiparkinsonian agents, antihypertensive agents, beta-adrenergic blocking agents, nutritional agents, antivirals, DNA fragments, nucleic acids, genetic material, oligonucleotides, radioisotopes, or combinations of these classes of compounds. To those skilled in the art, other drugs or biologically active agents that can be released in an aqueous environment can be utilized in the described delivery system. Also, various forms of the drugs or biologically active agents may be used. These include, without limitation, forms such as uncharged molecules, molecular complexes, salts, ethers, esters, amides, and other chemically modified forms of the biologically active agent which are biologically activated when injected into a body.

In a further embodiment, the sterile bioactive agent may also be added to the oil phase as a suspension to enhance the loading of the bioactive agent in the delivery composition. Whatever the final composition that is arrived at the process allows a sterile composition to be prepared. Also, the further behavior of the delivery composition in forming a delivery system and the subsequent release of the bioactive agent is as described in U.S. 20030049320 A1, AU 0222505 A5 and WO 02/49573 A3 to Bhagwatwar et al. and are all incorporated herein by reference.

The term “parenterally” as used herein is intended to include routes such as intramuscular, intravenous, subcutaneous, subdermal, intralesional, intratumoral, intracavitary, peritumoral, intraarticular, vaginal, intraperitoneal, intraabdominal, intrathecal, intraorgan and the like or on open wounds, fractures, ulcers, cancerous lesions and the like and is not to be construed as limiting on the scope of the invention. Thus, the composition can be used for the immediate or controlled release or both of bioactive agents or bioinactive agents wherever the use of a sterile composition is called for.

The following examples describe the invention in further detail and are not to be construed as limiting on the scope of the invention.

### EXAMPLES

Examples 1-5 describe different methods for preparation of sterile sorbitan monostearate. The same methods can be applied to the preparation of other sterile gelling agents including sorbitan monopalmitate.

### Comparative Example 1

Sorbitan monostearate (Sanyo, Japan) was subjected to sterilization by dry heat at a temperature of 180° C. for 2 hours. A dark brown colored mass was obtained. The material thus obtained though it would pass the test for sterility renders the final product unacceptable for pharmiaceutical use because of the dark brown color it imparts.

### Example 1

### Preparation of Sterile Sorbitan Monostearate by Direct Evaporation of the Solvent from a Solution in a Volatile Organic Solvent

Sorbitan monostearate (Sanyo, Japan) was dissolved in ethanol (50% w/v) by heating upto 60-65° C. in a water bath to form a clear solution. The hot solution was filtered through a 0.2 μm nylon 66 (Pall Gelman) sterilizing grade membrane filter under vacuum using a specially fabricated jacketed stainless steel filtration assembly, which was also maintained, at the elevated temperature. The filtrate was dried in a vacuum oven at 40° C. and 760 mm vacuum for 48 hours. The dried material was crushed and stored in a sterile container free from moisture.

### Example 2

### Preparation of Sterile Sorbitan Monostearate by Dissolving in a Hot Volatile Water-Miscible Solvent and Precipitating by Mixing with the same Solvent which is Cold

Sorbitan monostearate (Sanyo, Japan) was dissolved in ethanol (50% w/v) by heating upto 60-65° C. in a water bath to form a clear solution. The hot solution was filtered through a 0.2 μm nylon 66 (Pall Gelman) sterilizing grade membrane filter under vacuum using a specially fabricated jacketed stainless steel filtration assembly, which was also maintained, at the elevated temperature. The filtered solution was kept at 37° C. for 60minutes and was then slowly added into cold, sterile, particulate free ethanol at 2-8° C. The sorbitan monostearate precipitated as a fine powder at the bottom of the beaker. The precipitated sorbitan monostearate was collected and dried.

### Example 3

### Preparation of Sterile Sorbitan Monostearate by Dissolving in a Volatile Water-miscible Organic Solvent and Precipitating from a Non-Solvent

Sorbitan monostearate (Sanyo, Japan) was dissolved in ethanol (50% w/v) by heating upto 60-65° C. in a water bath to form a clear solution. The hot solution was filtered through a 0.2 μm nylon 66 (Pall Gelman) sterilizing grade membrane filter under vacuum using a specially fabricated jacketed stainless steel filtration assembly that was also maintained at the elevated temperature. The sorbitan monostearate was precipitated by drop-wise addition of the filtered ethanolic solution into sterile water for injection. The supernatant was decanted off and the precipitated sorbitan monostearate was collected on a filter paper and dried under vacuum at an elevated temperature. The dried material was crushed and stored in a sterile container free from moisture.

### Example 4

### Preparation of Sterile Sorbitan Monostearate by Dissolving in a Non-Volatile Water-Miscible Solvent and Precipitating from a Non-Solvent

Sorbitan monostearate (Sanyo, Japan) was dissolved in DMA (Fluka) by heating upto 60-65° C. in a water bath to form a clear solution. This solution was slowly added into sterile water for injection to cause precipitation of sorbitan monostearate, which was collected and dried.

### Example 5

### Sterilization by Gamma Irradiation

Sorbitan monostearate prepared in Example 1 was exposed to gamma irradiation at a dose of 25 KGy using a ⁶⁰Cobalt source at a contract irradiation laboratory. A dose of 25 KGy is accepted internationally to be sufficient to ensure sterility (British Pharmacopoeia). There was no change in appearance of the gelling agent.

These examples demonstrate that sterile sorbitan monostearate can be prepared by these techniques without any change in properties. The same techniques may be used to prepare sterile sorbitan monopalmitate. The further examples describe the use of the sterile sorbitan monostearate in the preparation of the gelled polymeric dispersions.

1. We claim a sterile gelling agent for use in a drug delivery system, which is substantially free from foreign particulate material, substantially free from moisture and substantially free from impurities.

2. A sterile gelling agent of claim 1, which retains its physico-chemical properties.

3. A sterile gelling agent of claim 1, belonging to the class of fatty acids, fatty alcohols, their metallic salts and their analogues.

4. A sterile gelling agent of claim 3 wherein the agent is either sorbitan monostearate or sorbitan monopalmitate.

5. A process for preparing a sterile gelling agent as in claim 1, comprising of
a. dissolution of the gelling agent in an organic solvent, followed by aseptic filtration of the solution and subsequent evaporation of solvent to obtain a dry product OR
b. dissolution of the gelling agent in a water-miscible organic solvent, followed by aseptic filtration of the solution, and subsequent precipitation or crystallization of the gelling agent from solution AND
c. Optionally, sterilizing the product obtained by either a or b by gamma irradiation.

6. A process for preparing a sterile gelling agent as in claim 5, where the aseptic filtration of the gelling agent is carried out at a temperature at which the gelling agents gels the solvent without undergoing degradation.

7. A process for preparing a sterile gelling agent as in claim 6, where the aseptic filtration of the gelling agent is carried out at a temperature at which the integrity of the filter membrane is not lost during aseptic filtration.

8. A process for preparing a sterile gelling agent as in claim 6, where the temperature used is below the boiling point of the organic solvent.

9. A process of claim 5 wherein an organic solvent is a volatile and water-miscible alkanol.

10. A process of claim 5 wherein the organic solvent is preferably ethyl alcohol.

11. A process of claim 5 wherein the organic solvent is either volatile or non volatile.

12. A process of claim 5 wherein the organic solvent is either water miscible or water immiscible.

13. A process of claim 5 wherein the organic solvent is volatile and is water miscible and is chosen from methylene chloride, ethyl acetate, benzene, petroleum ether, carbon tetrachloride or is non-volatile and is water miscible and is chosen from methanol, acetonitrile, acetone, ethanol, tetrahydrofuran, isopropanol.

14. The process of claim 5 wherein the organic solvent for the gelling agent is non-volatile and is water miscible and is chosen from N, N-dimethylacetamide, dimethylsulfoxide, N-methyl-2-pyrrolidone, triacetin, triethyl citrate, benzyl alcohol, propylene carbonate, decylmethylsulfoxide, dimethylformamide, glycofural, benzoyl benzoate, alkyl esters of aromatic acids, polyethylene glycols, propylene glycol, sorbitol and glycerol.

15. A process as in claim 5 wherein the concentration of the gelling agent in the solution for aseptic processing is from about 5 to about 80% w/w.

16. A process as in claim 5, wherein the filter used is of sterilizing grade.

17. A sterile gelling agent of claim 1 optionally sterilized by gamma radiation or obtained by an aseptic process.
